(19) United States
(12) Patent Application Publication
(10) Pub. No.: US 2021/0396766 A1
(43) Pub. Date: Dec. 23, 2021

(54) MARKERS OF RISK TO DEVELOP INSULIN RESISTANCE DURING CHILDHOOD AND YOUNG ADULTHOOD

(71) Applicant: SOCIETE DES PRODUITS NESTLE S.A., Vevey (CH)

(72) Inventors: Francois-Pierre Martin, Vuisternens-devant-Romont (CH); Jorg Hager, Houtaud (FR); Jonathan Pinkney, Plymouth Devon (GB); Joanne Hosking, Plymouth Devon (GB)

(21) Appl. No.: 17/279,700

(22) PCT Filed: Sep. 24, 2019
(86) PCT No.: PCT/EP2019/075638

§ 371 Date: Mar. 25, 2021

(30) Foreign Application Priority Data
Sep. 27, 2018 (EP) 18197378.5

(51) Int. Cl. G01N 33/68

(52) U.S. Cl. CPC G01N 33/6893; G01N 2800/042

(57) ABSTRACT

The present invention generally relates to a method for predicting high blood level glucose in biofluid of a subject. Methods of improving glucose level management in an adolescent subject are also provided. The present invention generally relates to a method for predicting high HOMA-IR in biofluid of a subject. Methods of improving glucose and insulin metabolism in a child, or an adolescent subject or a young adult are also provided.

### INTRODUCTION

With 1.8 billion adolescents worldwide (25% world pop.), and about 42 million children under 5 overweight or obese in 2013, management of pre-diabetes and Type-2 Diabetes (T2D) in childhood and adolescence has become critical. Nutrition has a pivotal role to play since both pre-diabetes and T2D are largely preventable and closely linked to lifestyle, dietary intake and exercise.

In addition, (Pre-)Diabetes in children differs from adults in many physiological and metabolic aspects, including insulin, sexual maturity & growth, neurologic vulnerability to hypoglycemia, and ability to provide self-care. However, compared to adult studies, there is less data in children, in whom insulin resistance (IR) is subject to marked variations, being particularly influenced by pubertal timing as well as both changing body composition and physical activity. In youths, the significance of pubertal IR is open to debate whilst the understanding of the underlying mechanisms that link obesity and IR is incomplete. Whereas IR relates to the resistance to insulin-mediated glucose uptake in insulin-sensitive tissues, childhood and pubertal IR may well result from various metabolic and physiological requirements, including the effects of increased growth hormone secretion (either direct and/or via the action of IGF-1) (Pinkney, Streeter et al. 2014).

In the context of metabolic health, childhood and adolescence, obesity introduces a significant disturbance into normal growth and pubertal patterns (Sandhu et al., 2006; Marcovecchio and Chiarelli, 2013). Recent analysis from the Earlybird study has demonstrated the important influences on IR of age and gender in puberty (Jeffery S et al. Pediatric Diabetes, 2017), which differs in many ways with the adult phenotype (Jeffery et al., 2012). The study exemplified how IR starts to rise in mid-childhood, some years before puberty, with more than 60% of the variation in IR prior to puberty remaining unexplained. In addition, conventional markers to detect diabetes, and to identify individuals at high risk of developing diabetes, and for adult metabolic disease risk, such as HbAlc, lose sensitivity and specificity for pediatric applications, suggesting that other factors influence the variance of these markers in youths (Hosking et al., 2014).

One potentially important factor currently being studied is the role of excess body weight during childhood. This can also influence pubertal development through effects on timing of pubertal onset and hormone levels (Marcovecchio and Chiarelli, 2013). The interactions of adiposity with puberty is complex and gender-specific. Moreover, in girls, higher level of IR limit further gain in body fat in the long term—an observation potentially consistent with the concept of IR as a mechanism of insulin desensitization as an adaptive response to weight gain (Hosking et al., 2011). Recently, weight gain and impaired glucose metabolism were shown to be predicted by inefficient subcutaneous fat cell lipolysis (Arner, Andersson et al. 2018). Adipocyte mobilization of fatty acids (lipolysis) is instrumental for energy expenditure. Lipolysis displays both spontaneous (basal) and hormone-stimulated activity. Thus, inefficient lipolysis (high basal/low stimulated) is linked to future weight gain and impaired glucose metabolism and may constitute a treatment target.

The role of resting energy expenditure and weight gain in children is subject to controversy, with particular interest in studying the influence of puberty on long term body composition. Obesity develops when energy intake is greater than energy expenditure, the excess energy being stored mainly as fat in adipose tissue. Body weight loss and prevention of weight gain can be achieved by reducing energy intake or bioavailability, increasing energy expenditure, and/or reducing storage as fat. However, overweight subjects or subjects at risk of becoming overweight often need nutritional assistance for better managing their body weight, e.g. through increasing satiety and/or reducing body weight gain.

Obesity is strongly associated with the development of IR, and there are strong associations between adiposity, IR, impaired glucose regulation and the development of type 2 diabetes in both adults and children. However, not all individuals who are obese develop diabetes and understanding of the underlying mechanisms which link obesity and IR remains incomplete. While it is broadly accepted that diabetes results from the combination of insulin secretory failure and/or IR, the accurate measurement of insulin secretion and IR in humans in-vivo is problematic. The most sensitive methods for such measurements (eg hyperglycemic clamp, euglycaemic hyperinsulinaemic clamp, or multipoint oral glucose tolerance tests) are not well suited to long term prospective studies with repeat measures and they are generally viewed as far too invasive for repeated use in children. Thus, there is a need for far simpler alternatives. The identification of novel metabolic biomarkers has the potential not only to more accurately identify individuals at risk of diabetes than simple measures of obesity or the more complex measures of insulin secretion and action, but also to further elucidate the mechanisms by which obesity and IR are linked.

To address these particular evidence gaps, the EarlyBird study was designed as a longitudinal cohort study of healthy children with the express intent to investigate the influences of anthropometric, clinical and metabolic processes on glucose and insulin metabolism during childhood and adolescence. The EarlyBird cohort is a non-interventional prospective study of 300 healthy UK children followed-up annually throughout childhood. The investigators tackled the challenging task of integrating and correlating the temporal variations of these different data types in the Earlybird childhood cohort from age 5 to age 20, including anthropometric, clinical and serum biomarker (metabonomic) data.

### Definitions

Various terms used throughout the specification are defined as shown below.

The following terms are used throughout the specification to describe the different early life stages of a subject of the invention, particularly a human subject:
Infant, Newborn: a human subject during the first month after birth;
Infant: a human subject between 1 and 23 months of age inclusive;
Child, Preschool: a human subject between the ages of 2 and 5 inclusive;
Child: a human subject between the ages of 6 and 12 inclusive;
Prepuberty: age 6 or 7 of a human subject;
Mid-childhood: age 7 or 8 of a human subject; and
Adolescent (or adolescence): a human subject between the ages of 13 and 18 inclusive (the corresponding early life stage in other subjects, for example in dogs, would be between the ages 6 months to 18 months inclusive)
Adulthood: 19 years old and above

The various metabolites mentioned throughout the specification are also known by other names. For example, the metabolite “3-D-hydroxybutyrate” is also known as (R)-(−)-beta-Hydroxybutyric acid; (R)-3-Hydroxybutanoic acid; 3-D-Hydroxybutyric acid; D-3-Hydroxybutyric acid; (R)-(−)-b-Hydroxybutyrate; (R)-(−)-b-Hydroxybutyric acid; (R)-(−)-beta-Hydroxybutyrate; (R)-(−)-β-hydroxybutyrate; (R)-(−)-β-hydroxybutyric acid; (R)-3-Hydroxybutyrate; (R)-3-Hydroxybutanoate; 3-D-Hydroxybutyrate; D-3-Hydroxybutyrate; 3-delta-Hydroxybutyrate; 3-delta-Hydroxybutyric acid; BHIB; D-(−)-3-Hydroxybutyrate; D-beta-Hydroxybutyrate; delta-(−)-3-Hydroxybutyrate; delta-3-Hydroxybutyrate; delta-3-Hydroxybutyric acid; and delta-beta-Hydroxybutyrate.

The metabolite “citrate” is also known as citric acid; 2-Hydroxy-1,2,3-propanetricarboxylic acid; 2-Hydroxytricarballylic acid; 3-Carboxy-3-hydroxypentane-1,5-dioic acid; 2-Hydroxy-1,2,3-propanetricarboxylate; 2-Hydroxytricarballylate; 3-Carboxy-3-hydroxypentane-1,5-dioate; beta-Hydroxytricarballylate; beta-Hydroxytricarballylic acid.

The metabolite “lactate” is also known as L-lactic acid; (+)-Lactic acid; (S)-(+)-Lactic acid; (S)-2-Hydroxypropanoic acid; (S)-2-Hydroxypropionic acid; L-(+)-alpha-Hydroxypropionic acid; L-(+)-Lactic acid; L-(+)-α-hydroxypropionate; (S)-2-Hydroxypropanoate; 1-Hydroxyethane 1-carboxylate; Milk acid; Sarcolactic acid; D-Lactic acid.

The metabolite “creatine” is also known as ((amino(imino)Methyl)(methyl)amino)acetic acid; (alpha-methylguanido)Acetic acid; (N-methylcarbamimidamido)Acetic acid; alpha-methylguanidino Acetic acid; Methylglycocyamine; N-(Aminoiminomethyl)-N-methylglycine; N-[(e)-amino(imino)METHYL]-N-methylglycine; N-Amidinosarcosine; N-Carbamimidoyl-N-methylglycine; N-Methyl-N-guanylglycine; (α-methylguanido)acetate; Methylguanidoacetate; [[amino(imino)Methyl](methyl)amino]acetate; (N-methylcarbamimidamido)Acetate.

The metabolite “Histidine” is also known as S)-4-(2-amino-2-Carboxyethyl)imidazole; (S)-alpha-amino-1H-Imidazole-4-propanoic acid; (S)-alpha-amino-1H-Imidazole-4-propionic acid; (S)-1H-Imidazole-4-alanine; (S)-2-amino-3-(4-Imidazolyl)propionsaeure; (S)-Histidine; (S)1H-Imidazole-4-alanine; 3-(1H-Imidazol-4-yl)-L-alanine; amino-1H-Imidazole-4-propanoate; amino-1H-Imidazole-4-propanoic acid; amino-4-lmidazoleproprionate; amino-4-Imidazoleproprionic acid; Glyoxaline-5-alanine.

The metabolite “Glycine” is also known as Aminoacetic acid; Aminoessigsaeure; Aminoethanoic acid; Glycocoll; Glykokoll; Glyzin; Leimzucker; 2-Aminoacetate; amino-Acetic acid; Glicoamin; Glycolixir; Glycosthene; Gyn-hydralin; Padil HMDB

The metabolite “Lysine” is also known as (S)-2,6-Diaminohexanoic acid; (S)-alpha,epsilon-Diaminocaproic acid; (S)-Lysine; 6-ammonio-L-Norleucine; L-2,6-Diaminocaproic acid; L-Lysin; Lysina; Lysine acid; Lysinum; (S)-2,6-Diaminohexanoate; (+)-S-Lysine; (S)-2,6-diamino-Hexanoate; (S)-2,6-diamino-Hexanoic acid; (S)-a,e-Diaminocaproate; (S)-a,e-Diaminocaproic acid; 2,6-Diaminohexanoate; 2,6-Diaminohexanoic acid; 6-amino-Aminutrin; 6-amino-L-Norleucine; a-Lysine; alpha-Lysine; Aminutrin; L-2,6-Diainohexanoate; L-2,6-Diainohexanoic acid; Enisyl MeSH.

The metabolite “Arginine” is also known as (2S)-2-amino-5-(carbamimidamido)Pentanoic acid; (2S)-2-amino-5-Guanidinopentanoic acid; (S)-2-amino-5-Guanidinopentanoic acid; (S)-2-amino-5-Guanidinovaleric acid; L-(+)-Arginine; (S)-2-amino-5-[(Aminoiminomethyl)amino]-pentanoate; (S)-2-amino-5-[(Aminoiminomethyl)amino]-pentanoic acid; (S)-2-amino-5-[(Aminoiminomethyl)amino]pentanoate; (S)-2-amino-5-[(Aminoiminomethyl)amino]pentanoic acid; 2-amino-5-Guanidinovalerate; 2-amino-5-Guanidinovaleric acid; 5-[(Aminoiminomethyl)amino]-L-norvaline; L-a-amino-D-Guanidinovalerate; L-a-amino-D-Guanidinovaleric acid; L-alpha-amino-delta-Guanidinovalerate; L-alpha-amino-delta-Guanidinovaleric acid; N5-(Aminoiminomethyl)-L-ornithine.

The term Insulin resistance (IR) is a pathological condition in which cells fail to respond normally to the hormone insulin. The body produces insulin when glucose starts to be released into the bloodstream from the digestion of carbohydrates (primarily) in the diet. Under normal conditions of insulin reactivity, this insulin response triggers glucose being taken into body cells, to be used for energy, and inhibits the body from using fat for energy, thereby causing the concentration of glucose in the blood to decrease as a result, staying within the normal range even when a large amount of carbohydrates is consumed. During insulin resistance, however, excess glucose is not sufficiently absorbed by cells even in the presence of insulin, thereby causing an increase in the level of blood sugar. IR is one of the factors involved in type 2 Diabetes and Pre-diabetes.

IR can be diagnosed through different means:
Fasting insulin levels: A fasting serum insulin level greater than 25 mIU/L or 174 pmol/L is considered insulin resistance
Glucose tolerance test and Matsuda index
Homeostatic Model Assessment (HOMA), the normal reference range for HOMA-IR differs depending on ethnicity and gender, and must be defined for each population.
Quantitative insulin sensitivity check index (QUICKI).
Hyperinsulinemic euglycemic clamp
Modified insulin suppression test

The term “pre-diabetes” describes a condition in which fasting blood glucose levels are equal or higher than 5.6 mmol/L of blood plasma, although not high enough to be diagnosed with type 2 diabetes. Pre-diabetes has no signs or symptoms. People with pre-diabetes have a higher risk of developing type 2 diabetes and cardiovascular (heart and circulation) disease. Without sustained lifestyle changes, including healthy eating, increased activity and losing weight, approximately one in three people with pre-diabetes will go on to develop type 2 diabetes. There are two pre-diabetic conditions:
Impaired glucose tolerance is defined as two-hour glucose levels of 140 to 199 mg per dL (7.8 to 11.0 mmol) on the 75-g oral glucose tolerance test. Levels for diabetes are therefore above 11 mmol in ogtt.
Impaired fasting glucose (IFG) is where blood glucose levels are escalated in the fasting state but not high enough to be classified as diabetes. Impaired fasting glucose is defined as glucose levels of 100 to 125 mg per dL (5.6 to 6.9 mmol per L) in fasting patients. Levels for diabetes are therefore above 6.9 mmol.
It is possible to have both Impaired Fasting Glucose (IFG) and Impaired Glucose Tolerance (IGT).

As used herein, the term “reference value” can be defined as the average value measured in biofluid samples of a substantially healthy normal glycemic population. Said population may have an average fasting glucose level of less than 5.6 mmol/L. The average age of said population is preferably substantially the same as that of the subject. The average BMI sds of said population is preferably substantially the same as that of the subject. The average physical activity level of said population is preferably substantially the same as that of the subject. Said population may be of substantially the same race as the human subject. Said population may number at least 2, 5, 10, 100, 200, 500, or 1000 individuals. Said population may be substantially the same breed when the subject is a pet.

The term “high levels of glucose” or “high glucose levels” is defined as equal to or higher than 5.6 mmol/L as measured in a biofluid sample of a subject.

The term “biofluid” can be, for example, human blood (particularly human blood serum, human blood plasma), urine or interstitial fluids.

“Overweight” is defined for an adult human as having a BMI between 25 and 30. “Body mass index” or “BMI” means the ratio of weight in kg divided by the height in metres, squared. “Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30. “Normal weight” for an adult human is defined as a BMI of 18.5 to 25, whereas “underweight” may be defined as a BMI of less than 18.5. Body mass index (BMI) is a measure used to determine childhood overweight and obesity in children and teens. Overweight in children and teens is defined as a BMI at or above the 85th percentile and below the 95th percentile for children and teens of the same age and sex. Obesity is defined as a BMI at or above the 95th percentile for children and teens of the same age and sex. Normal weight in children and teens is defined as a BMI at or above the 5th percentile and below the 85th percentile for children and teens of the same age and sex. Underweight in children and teens is defined as below the 5th percentile for children and teens of the same age and sex. BMI is calculated by dividing a person's weight in kilograms by the square of height in meters. For children and teens, BMI is age- and sex-specific and is often referred to as BMI-for-age. A child's weight status is determined using an age- and sex-specific percentile for BMI rather than the BMI categories used for adults. This is because children's body composition varies as they age and varies between boys and girls. Therefore, BMI levels among children and teens need to be expressed relative to other children of the same age and sex.

The term “subject” is preferably a human subject or can be a pet subject e.g. a cat a dog.

The term “substantially” is taken to mean 50% or greater, more preferably 75% or greater, or more preferably 90% or greater. The term “about” or “approximately” when referring to a value or to an amount or percentage is meant to encompass variations of in some embodiments ±20%, in some embodiments ±10%, in some embodiments ±5%, in some embodiments ±1%, in some embodiments ±0.5%, and in some embodiments ±0.1% from the specified value, amount or percentage.

### DETAILED DESCRIPTION

The present invention provides a method for predicting insulin resistance (IR) in a subject, said method comprising:

a. (i) determining the levels of lactate and histidine, and one or more of citrate, 3-D-hydroxybutyrate, lysine, glycine, and creatine in a biofluid sample of said subject; or (ii) determining the levels of one or more of lactate, histidine, citrate, 3-D-hydroxybutyrate, lysine, glycine, and creatine in a biofluid sample of said subject;

b. comparing the levels of one or more of lactate, histidine, creatine:glycine ratio, citrate, 3-D-hydroxybutyrate, lysine with a reference value;

c. identifying the subject as being at high risk of IR if
(I) the levels of one or more of lactate, creatine:glycine ratio are higher than the reference value in b; and/or
(II) the levels of one or more of histidine, citrate, 3-D-hydroxybutyrate, lysine are lower than the reference value in b;

The present invention further provides a method for predicting IR in a subject, said method comprising:

a. (i) determining the levels of lactate and histidine, and one or more of citrate, 3-D-hydroxybutyrate, lysine, glycine, and creatine in a biofluid sample collected from said subject being a child or an adolescent; and/or (ii) determining the levels of one or more of lactate, histidine, citrate, 3-D-hydroxybutyrate, lysine, glycine, and creatine in a biofluid sample collected from said subject being a child or an adolescent;

b. comparing the levels of one or more of lactate, histidine, creatine:glycine ratio, citrate, 3-D-hydroxybutyrate, lysine with a reference value;

c. identifying the subject as being at high risk of IR in adolescence and/or adulthood if
(I) the levels of one or more of lactate, creatine:glycine ratio are higher than the reference value in b; and/or
(II) the levels of one or more of histidine, citrate, 3-D-hydroxybutyrate, lysine are lower than the reference value in b;

In one embodiment, the method for predicting IR in a subject comprises:

a. determining the level of lactate in the biofluid sample collected from said subject being a child or an adolescent;

b. comparing the level of lactate with a reference value;

c. identifying the subject as being at high risk of high IR in adolescence and/or adulthood if the level of lactate is higher than the reference value in b;

In one embodiment, the method for predicting IR in a subject comprises:

a. determining the levels of glycine and creatine in the biofluid sample collected from said subject being a child or an adolescent;

b. comparing the level of creatine:glycine ratio with a reference value;

c. identifying the subject as being at high risk of IR in adolescence and/or adulthood if the level of creatine:glycine ratio is higher than the reference value in b;

In one embodiment, the method for predicting IR in a subject comprises:

a. determining the level of histidine in the biofluid sample collected from said subject being a child or an adolescent;

b. comparing the level of histidine with a reference value;

c. identifying the subject as being at high risk of IR in adolescence and/or adulthood if the level of histidine is lower than the reference value in b;

The present invention further provides a method for predicting IR in a subject, said method comprising:

a. (i) determining the levels of lactate and histidine, and one or more of citrate, 3-D-hydroxybutyrate, lysine, glycine, and creatine in a biofluid sample collected from said subject being a child; and/or (ii) determining the levels of one or more of lactate, histidine, citrate, 3-D-hydroxybutyrate, lysine, glycine, and creatine in a biofluid sample collected from said subject being a child;

b. comparing the levels of one or more of lactate, histidine, creatine:glycine ratio, citrate, 3-D-hydroxybutyrate, lysine with a reference value;

c. identifying the subject as being at high risk of IR in adolescence if

(I) the levels of one or more of lactate, creatine:glycine ratio are higher than the reference value in b; and/or

(II) the levels of one or more of histidine, citrate, 3-D-hydroxybutyrate, lysine are lower than the reference value in b;

The present invention further provides a method for predicting IR in a subject, said method comprising:

a. (i) determining the levels of lactate and histidine, and one or more of citrate, 3-D-hydroxybutyrate, lysine, glycine, and creatine in a biofluid sample collected from said subject being a child; and/or (ii) determining the levels of one or more of lactate, histidine, citrate, 3-D-hydroxybutyrate, lysine, glycine, and creatine in a biofluid sample collected from said subject being a child;

b. comparing the levels of one or more of lactate, histidine, creatine:glycine ratio, citrate, 3-D-hydroxybutyrate, lysine with a reference value;

c. identifying the subject as being at high risk of IR in adulthood if

(I) the levels of one or more of lactate, creatine:glycine ratio are higher than the reference value in b; and/or

(II) the levels of one or more of histidine, citrate, 3-D-hydroxybutyrate, lysine are lower than the reference value in b;

The present invention further provides a method for predicting IR in a subject, said method comprising:

a. (i) determining the levels of lactate and histidine, and one or more of citrate, 3-D-hydroxybutyrate, lysine, glycine, and creatine in a biofluid sample collected from said subject being an adolescent; and/or (ii) determining the levels of one or more of lactate, histidine, citrate, 3-D-hydroxybutyrate, lysine, glycine, and creatine in a biofluid sample collected from said subject being an adolescent;

b. comparing the levels of one or more of lactate, histidine, creatine:glycine ratio, citrate, 3-D-hydroxybutyrate, lysine with a reference value;

c. identifying the subject as being at high risk of IR in adolescence if

(I) the levels of one or more of lactate, creatine:glycine ratio are higher than the reference value in b; and/or

(II) the levels of one or more of histidine, citrate, 3-D-hydroxybutyrate, lysine are lower than the reference value in b;

The present invention further provides a method for predicting IR in a subject, said method comprising:

a. (i) determining the levels of lactate and histidine, and one or more of citrate, 3-D-hydroxybutyrate, lysine, glycine, and creatine in a biofluid sample collected from said subject being an adolescent; and/or (ii) determining the levels of one or more of lactate, histidine, citrate, 3-D-hydroxybutyrate, lysine, glycine, and creatine in a biofluid sample collected from said subject being an adolescent;

b. comparing the levels of one or more of lactate, histidine, creatine:glycine ratio, citrate, 3-D-hydroxybutyrate, lysine with a reference value;

c. identifying the subject as being at high risk of IR in adulthood if

(I) the levels of one or more of lactate, creatine:glycine ratio are higher than the reference value in b; and/or

(II) the levels of one or more of histidine, citrate, 3-D-hydroxybutyrate, lysine are lower than the reference value in b;

In one embodiment, the levels of lactate, histidine, creatine, glycine and one or more of citrate, 3-D-hydroxybutyrate, lysine, in a biofluid sample collected from said subject in step a(i) are determined.

In one embodiment, the levels of lactate, histidine, creatine, glycine and two or more of citrate, 3-D-hydroxybutyrate, lysine in a biofluid sample collected from said subject in step a(i) are determined.

In one embodiment, the levels of lactate, histidine, creatine, glycine, citrate, 3-D-hydroxybutyrate, lysine in a biofluid sample collected from said subject in step a(i) are determined.

In one embodiment, high IR corresponds to HOMA-IR values equal to or higher than 1.5.

In one embodiment, high IR corresponds to HOMA-IR values equal to or higher than 2.

In one embodiment, IR is severe, corresponding to HOMA-IR values equal to or higher than 5.

In one embodiment, said subject is not overweight when said biofluid sample is collected.

In one embodiment, said subject is not obese when said biofluid sample is collected.

In an alternative embodiment, the present invention provides a method for predicting HOMA-IR below 1.5 in a subject, said method comprising:

a. (i) determining the levels of lactate and histidine, and one or more of citrate, 3-D-hydroxybutyrate, lysine, glycine, and creatine in the biofluid sample collected from said subject being a child or an adolescent; and/or (ii) determining the levels of one or more of lactate, histidine, citrate, 3-D-hydroxybutyrate, lysine, glycine, and creatine in the biofluid sample collected from said subject being a child or an adolescent;

b. comparing the levels of one or more of lactate, histidine, creatine:glycine ratio, citrate, 3-D-hydroxybutyrate, lysine with a reference value;

c. identifying the subject as being at high risk of IR in adolescence and/or adulthood if
(I) the levels of one or more of lactate, creatine:glycine ratio are higher than the reference value in b; and/or
(II) the levels of one or more of histidine, citrate, 3-D-hydroxybutyrate, lysine are lower than the reference value in b;

In one embodiment, said biofluid sample collections are taken from the subject at age 5 years, 6 years, 7 years, 8 years, 9 years, 10 years, 11 years, 12 years, 13 years, 14 years, 15 years, 16 years, 17 years, or 18 years separated by at least a one year interval.

In one aspect of the invention, the biofluid sample is collected when the subject is age 5 years.

In one aspect of the invention, the biofluid sample is collected when the subject is age 6 years.

In one aspect of the invention, the biofluid sample is collected when the subject is age 7 years.

In one aspect of the invention, more than one biofluid sample is collected from said subject in steps a(i) and/or a(ii).

In one aspect of the invention, metabolite measurements are made by NMR (Nuclear Magnetic Resonance). Alternatively, metabolite measurements may be made by mass spectroscopy or by clinical assay.

In one aspect of the invention, the age sub-range of 13 to 16 years inclusive is chosen as being representative of adolescence.

In one aspect of the invention, the age 15 years is chosen as being representative of adolescence.

In one aspect of the invention, the age 20 years is chosen as being representative of adulthood.

In one aspect of the invention, the reference value is a predetermined standard.

In one aspect of the invention, the biofluid sample is human blood serum.

The present invention also provides a method of improving glucose level management in a child or an adolescent subject comprising (i) predicting whether said subject has IR according to the invention; and (ii) providing a method of modifying the lifestyle of a subject identified as being at high risk of having insulin resistance in adolescence and/or adulthood, wherein said dietary intervention enhances insulin sensitivity, reduces likelihood, lowers, or prevents insulin resistance and/or reduces the glucose level.

In one aspect of the invention, said modification of lifestyle lowers insulin resistance. In one aspect of the invention, said modification of lifestyle prevents insulin resistance. In one aspect of the invention, said modification of lifestyle prevents insulin resistance.

In one aspect of the invention, said modification of lifestyle is provided through prepuberty and/or puberty.

In one aspect of the invention, said method reduces the likelihood or prevents the onset of one or more metabolic disorders, particularly type 2 diabetes, particularly in early adulthood.

In one aspect of the invention, said modification of lifestyle is provided through prepuberty, puberty, and adolescence.

In one aspect of the invention, the modification in lifestyle in the subject comprises a change in diet, preferably comprising administering at least one nutritional product to the subject that is part of a diet that modulates levels of glucose

In one aspect of the invention, administering at least one nutritional product to the subject that is part of a diet that modulates levels of glucose promotes a reduction in glucose or prevents an increase in glucose levels in the subject.

In one aspect of the invention, the change in diet comprises a decreased consumption of fat and/or an increase in consumption of low fat foods such that not more than 20% of daily calories are obtained from fat.

Low fat foods includes bread and flour, oats, breakfast cereals, wholegrain rice and pasta, fresh, frozen and tinned vegetables and fruits, dried beans and lentils, baked or boiled potatoes, dried fruits, white fish, shellfish, lean white meat such as chicken and turkey breast without skin, skimmed and smi skimmed milk, cottage or curd cheese, low fat yoghourt, or egg whites. Most adults get 20%-35% of their daily calories from fat. That equates to about 44 to 77 grams of fat a day if 2,000 calories a day are consumed. Low fat foods can also be selected from wholemeal flour and bread, porridge oats, high-fibre breakfast cereals, dried beans and lentils, walnuts, herring, mackerel, sardines, kippers, pilchards, salmon and lean white meat.

In one aspect of the invention, the change in diet comprises a ketogenic type of diet that provides sufficient protein for body growth and repair, and sufficient calories to maintain the correct weight for age and height.

A ketogenic diet may be achieved by excluding high-carbohydrate foods such as starchy fruits and vegetables, bread, pasta, grains and sugar, while increasing the consumption of foods high in fat such as nuts, cream and butter. A variant of the classic diet known as the medium-chain triglycerides (MCT) ketogenic diet uses a form of coconut oil, which is rich in MCTs, to provide around half the calories. As less overall fat is needed in this variant of the diet, a greater proportion of carbohydrate and protein can be consumed, allowing a greater variety of food choices. In one aspect of the invention, the change in diet comprises a change to a ketogenic diet. In one embodiment, a ketogenic diet is the consumption of under 20 g of carbohydrates per day.

In one aspect of the invention, the change in diet comprises a change to a Mediterranean diet.

In one embodiment, said Mediterranean diet is higher in fat, that may include intermittent fasting. For instance, in typical Mediterranean countries breakfast may be skipped, and a big lunch may be taken with equal number of calories as breakfast and lunch.

A Mediterranean diet typically contains three to nine servings of vegetables, half to two servings of fruit, one to 13 servings of cereals and up to eight servings of olive oil daily. In one embodiment, it contains approximately not less than 9300 kJ. In one embodiment, it contains not more than 37% as total fat (particularly not less than 18% as monounsaturated and not more than 9% as saturated). In one embodiment, it contains not less than 33 g of fibre per day.

As an example, food type and intake, as well as nutrient content of the Mediterranean diet are described by Davis et al. (Reference Definition of the Mediterranean Diet: A Literature Review, Davis et al., Nutrients, 7(11), 9139-9153, 2015);

In one aspect of the invention, the change in diet comprises a change to a moderate low carbohydrate diet, to maintain or reach normal blood sugar levels throughout the day. In one embodiment, a moderate low carbohydrate diet is the consumption of between 20 g to 50 g of carbohydrates per day. By comparison, a standard diet is consumption of about 50 g to 100 g of carbohydrates per day.

In one aspect of the invention, the change in diet comprises a change to a vegan diet. Typically, a vegan diet is well-balanced in macronutrient and micronutrient composition and results in lower average blood sugar levels throughout the day. Vegan diets are plant-based diet regimens that exclude meat, eggs, dairy products, and any other animal-derived foods and ingredients.

In contrast, a vegetarian diet emphasizes plant-based foods but can also include dairy, eggs, honey, and fish. Both vegan and vegetarian diets can be healthful for all life stages with appropriate selection of plant-based foods that adequately meet nutrition requirements for protein, iron, n-3 fatty acids, iodine, zinc, calcium, and vitamin B12. An intermittent vegan diet regimen that is alternated within a habitual, balanced omnivorous diet can also meet these nutritional requirements.

In one aspect of the invention, the change in diet comprises a supplementation of essential nutrients aiming at improving glucose management, such as essential amino acids, lipid and water soluble vitamins, minerals, or a combination of nutrients.

Examples of essential nutrients are amino acids (phenylalanine, valine, threonine, tryptophan, methionine, leucine, isoleucine, lysine, and histidine); fatty acids (alpha-linolenic acid (omega-3 fatty acid) and linoleic acid (omega-6 fatty acid); vitamins (vitamin A, Bs (1-12), Vitamin C, Vitamin D, Vitamin E); minerals such as “major minerals” (calcium, phosphorus, potassium, sodium, chlorine, and magnesium) and “minor minerals” (metals such as iron, zinc, manganese and copper); and conditional nutrients (choline, inositol, taurine, arginine, glutamine and nucleotides).

In one aspect of the invention, the change in diet is associated with physical activity program management. The physical activity program should be adapted to body composition, medical conditions and age of the subjects, aiming at weight loss or weight management, and improvement of body fat mass and lean mass for optimal glucose management outcome.

For instance, the solution may be part of a Physical Activity Program which use all opportunities for students to be physically active, meet the nationally-recommended minutes of physical activity each day (e.g. 60 minutes of moderate to vigorous physically activity each day). For instance, the program may follow public health guidelines for physical activity for children and young people (as an example, National institute for health and care excellence, UK: https://www.nice.org.uk/guidance).

One aspect of the invention further comprises a step of repeating the step of predicting levels of IR in a subject after modifying the lifestyle of the subject.

The present invention also provides a kit of parts comprising means to measure levels of lactate, histidine, citrate, 3-D-hydroxybutyrate, lysine, glycine, and creatine in biofluid of a subject in prepuberty.

The present invention also provides the use of a kit of parts according to the invention, to predict a subject in prepuberty of having IR or developing prediabetes in adolescence and/or adulthood.

### EXAMPLES

### Example 1

### Methods Used During the Study

### Study Population

The EarlyBird Diabetes Study incorporates a 1995/1996 birth cohort recruited in 2000/2001 when the children were 5 years old (307 children, 170 boys). The collection of data from the Early Bird cohort is composed of several clinical and anthropometric variables measured on an annual basis from the age of 5 to the age of 16. The study was conducted in accordance with the ethics guidelines of the Declaration of Helsinki II; ethics approval was granted by the Plymouth Local Research Ethics Committee (1999), and parents gave written consent and children verbal assent.

Following a good cohort retention at an age when some young people will begin moving from home to start their own lives. A follow-up study was prepared from June 2013 and began study visits in February 2015 until summer 2016. A total of 178 Earlybird participants completed this follow-up visit as an adult (average age of 20 years old) where data were collected using an adapted study protocol.

### Anthropometric Parameters

BMI was derived from direct measurement of height (Leicester Height Measure; Child Growth Foundation, London, U.K.) and weight (Tanita Solar 1632 electronic scales), performed in blind duplicate and averaged. BMI SD scores were calculated from the British 1990 standards.

Physical activity was measured annually from 5 years by accelerometry (Acti-Graph [formerly MTI/CSA]). Children were asked to wear the accelerometers for 7 consecutive days at each annual time point, and only recordings that captured at least 4 days were used.

Resting energy expenditure was measured by indirect calorimetry using a ventilated flow through hood technique (Gas Exchange Measurement, Nutren Technology Ltd, Manchester, UK). Performance tests reportedly show a mean error of 0.3±2.0% in the measurement of oxygen consumption and 1.8±1% in that of carbon dioxide production. Measurements were performed in a quiet thermoneutral room (20° C.) after overnight fasting period of at least 6 hours, to minimize any effect attributable to the thermic effect of food. Data were collected for a minimum of 10 minutes and the respiratory quotient (RQ) was calculated as an indicator of basal metabolic rate (BMR).

### Clinical Parameters

Peripheral blood was collected annually into EDTA tubes after an overnight fast and stored at −80° C. Insulin resistance (IR) was determined each year from fasting glucose (Cobas Integra 700 analyzer; Roche Diagnostics) and insulin (DPC IMMULITE) (cross-reactivity with proinsulin, 1%) using the homeostasis model assessment program (HOMA-IR), which has been validated in children.

### Serum Metabonomics

400 μL of blood serum were mixed with 200 μL of deuterated phosphate buffer solution 0.6 M KH2PO4, containing 1 mM of sodium 3-(trimethylsilyl)-[2,2,3,3-2H4]-1-propionate (TSP, chemical shift reference 6H=0.0 ppm). 550 μL of the mixture were transferred into 5 mm NMR tubes.

1H NMR metabolic profiles of serum samples were acquired with a Bruker Avance III 600 MHz spectrometer equipped with a 5 mm cryoprobe at 310 K (Bruker Biospin, Rheinstetten, Germany) and processed using TOPSPIN (version 2.1, Bruker Biospin, Rheinstetten, Germany) software package as reported previously. Standard ¹H NMR one-dimensional pulse sequence with water suppression, Carr-Purcell-Meiboom-Gill (CPMG) spin-echo sequence with water suppression, and diffusion-edited sequence were acquired using 32 scans with 98K data-points. The spectral data (from δ 0.2 to δ 10) were imported into Matlab software with a resolution of 22K data-points (version R2013b, the Mathworks Inc, Natwick Mass.) and normalized to total area after solvent peak removal. Poor quality or highly diluted spectra were discarded from the subsequent analysis.

1H-NMR spectrum of human blood plasma enables the monitoring of signals related to lipoprotein bound fatty acyl groups found in triglycerides, phospholipids and cholesteryl esters, together with peaks from the glyceryl moiety of triglycerides and the choline head group of phosphatidylcholine. This data also covers quantitative profiling of major low molecular weight molecules present in blood. Based on internal database, representative signals of metabolites assignable on 1H CPMG NMR spectra were integrated, including asparagine, leucine, isoleucine, valine, 2-ketobutyric acid, 3-methyl-2-oxovaleric acid, alpha-ketoisovaleric acid, (R)-3-hydroxybutyric acid, lactic acid, alanine, arginine, lysine, acetic acid, N-acetyl glycoproteins, 0-acetyl glycoproteins, acetoacetic acid, glutamic acid, glutamine, citric acid, dimethylglycine, creatine, citrulline, trimethylamine, trimethylamine N-oxide, taurine, proline, methanol, glycine, serine, creatinine, histidine, tyrosine, formic acid, phenylalanine, threonine, and glucose. In addition, in diffusion edited spectra, signals associated to different lipid classes were integrated, including phospholipids containing choline, VLDL subclasses, unsaturated and polyunsaturated fatty acid. The signals are expressed in arbitrary units corresponding to a peak area normalized to total metabolic profiles, which is representative of relative change in metabolite concentration in the serum.

### Mass-Spectrometry Based Determination of Serum Amino Acids

Blood serum amino acids were quantified on selected samples using an in-house automated quantification method of amino acids in human plasma and serum by UPLC-MS/MS. Briefly, following a step of precipitation, derivatization and dilution, samples are submitted to liquid chromatography (Acquity I-class, Waters) coupled to mass spectrometry analysis (Xevo TQ-XS triple quadrupole, Waters). For chromatographic separation, a gradient composed a mobile phase of Ammonium Formate (Ammonium formate 0.55 g/L in water at 0.1% formic acid), and a second mobile phase of acetonitrotion (acetonitrile 0.1% formic acid). Analyte concentrations are calculated from peaks area ratio of the compounds to their corresponding internal standards. Results are expressed in μM. Peaks are integrated using AA_quantitationmeth in TargetLynx functionality included in MassLynx software.

### Statistics

The distribution of the outcome variable, IR, was skewed and so log-transformed for analysis. For both pilot and main study analyses, using data at all ages simultaneously, mixed effects modelling was used to assess the association between IR (HOMA-IR) and individual metabolites, taking into account age, BMI sds, physical activity and pubertal timing (APHV). Random intercepts were included as well as age (categorized to allow for non-linear change in IR over time), gender, DEXA % fat, APHV, MVPA (number of minutes spent in moderate-vigorous physical activity) and individual metabolites (in separate models) as fixed effects.

The present inventors carried a first study on a sub-set of 40 of the participants from 5 y to 14 y (Pilot study), and assessed repeatability on another subset of 150 participants from 5 y to 16 y (Main study). The present inventors carried a first study on a sub-set of 40 of the participants from 5 y to 14 y (Pilot study), and assessed repeatability on another subset of 150 participants from 5 y to 16 y (Main study). In the Pilot study, 40 subjects were chosen on the basis of having a complete set of samples available for analysis at each time-point between 5 y and 14 y (20 boys), having been stratified by IR at 5 and 14 years. In the Main study, 150 subjects were chosen to include all of those who had shown impaired fasting glucose at one or more time-points during the course of the study. Only 28 children were common in the two studies. The subjects who had shown impaired fasting glucose were matched for gender resulting in the selection of 105 boys and 45 girls.

To assess further which IR-associated metabolite may be an early indicator of IR trajectories, the present inventors stratified the main study population according to low or high IR status over the 14-16 year age range. Arbitrarily the 91st centile for the HOMA-IR distribution was employed as a threshold to define children with high IR status. Here, mixed effects modelling was used to assess the association between IR and individual metabolites. Modelling was carried out in R software (www.R-project.org) using the Imer function in the package Ime4 (Bates, Maechler et al. 2015) and p-values calculated using the Satterthwaite approximation implemented in the ImerTest package (Kuznetsova, Brockhoff et al. 2016).

### Example 2

### Measurement of Metabolite Concentrations

Clinical and anthropometrics characteristics of the children in the pilot study at 5 and 14 years are summarized in Table 1 and those in the main study at 5 y, 14 y and 16 y in Table 2. In both genders there was a decrease in HOMA-IR up to around 8 y, which was followed by an increase through puberty, this trend being dependent on timing of peak height velocity (age*APHV interaction p<0.001). IR was also positively associated with BMI sds (p<0.001).

TABLE 1
Characteristics of the cohort at 5 y and 14 y by gender.

| | | Boys | | Girls | |
|---|---|---|---|---|---|
| Age (years) | 5 y | 5.1 | (4.8-5.3) | 4.8 | (4.7-5.0) |
| | 14 y | 13.9 | (13.6-14.1) | 13.8 | (13.7-14.1) |
| BMI sds | 5 y | −0.04 | (−0.50-0.72) | 0.40 | (0.04-0.80) |
| | 14 y | 0.43 | (−0.13-1.29) | 0.78 | (−0.05-1.48) |
| Moderate-vigorous | 5 y | 46.1 | (34.1-67.1) | 55.1 | (44.9-62.5) |
| physical activity | 14 y | 52.4 | (29.7-77.0) | 42.7 | (29.3-51.4) |
| (minutes/day) | | | | | |
| Age at peak height | | 13.4 | (12.9-13.8) | 11.9 | (11.1-12.5) |
| velocity (years) | | | | | |
| IR (HOMA2-IR) | 5 y | 0.47 | (0.37-0.84) | 0.85 | (0.34-1.02) |
| | 14 y | 1.25 | (0.56-1.63) | 0.98 | (0.78-2.23) |

Data are median (interquartile range)

TABLE 2
Characteristics of the main cohort at 5 y, 14 y and 16 y by gender.

| | | Boys | | Girls | |
|---|---|---|---|---|---|
| Age (years) | 5 y | 4.8 | (4.7-5.0) | 4.9 | (4.8-5.1) |
| | 14 y | 13.8 | (13.6-14.0) | 13.9 | (13.8-14.0) |
| | 16 y | 15.8 | (15.6-16.0) | 15.9 | (15.8-16.1) |
| BMI sds | 5 y | 0.09 | (−0.48-0.80) | 0.36 | (−0.49-1.16) |
| | 14 y | 0.32 | (−0.64-0.98) | 0.85 | (0.21-1.60) |
| | 16 y | 0.44 | (−0.31-1.32) | 0.70 | (0.03-1.60) |
| Moderate-vigorous | 5 y | 53.1 | (41.6-64.1) | 40.1 | (31.9-51.6) |
| physical activity | 14 y | 44.4 | (30.7-63.14) | 35.3 | (18.4-50.4) |
| (minutes/day) | 16 y | 43.9 | (22.5-58.6) | 27.0 | (16.8-40.9) |
| Age at peak height | | 13.0 | (12.8-13.4) | 11.6 | (10.8-12.3) |
| velocity (years) | | | | | |
| IR (HOMA2-IR) | 5 y | 0.49 | (0.22-0.74) | 0.76 | (0.60-1.00) |
| | 14 y | 1.00 | (0.79-1.47) | 1.47 | (1.00-1.70) |
| | 16 y | 0.70 | (0.23-1.16) | 0.84 | (0.23-1.18) |

Data are median (interquartile range)

Using data at all ages simultaneously, mixed effects modelling was applied to assess the association between HOMA-IR and individual metabolites. In the pilot study several metabolites including BCAAs, lipids and other amino acids showed a significant association (p<0.05) with HOMA-IR in longitudinal models, independently of BMI sds, physical activity and pubertal timing as shown in Table 3. The Table 4 reports the outcomes of the same analysis conducted on the main study cohort.

TABLE 3
Estimates and p-values from mixed effects models
examining the association between metabolites and
insulin resistance in the pilot study (n = 40)

| | Metabolic | | | |
|---|---|---|---|---|
| Metabolite | pathway | Coef | SE | p-value |
| Citrate | Glycolysis | −0.132 | 0.028 | 0.000003 |
| | related | | | |
| Phospholipids | Lipid | −0.131 | 0.031 | 0.000038 |
| 3-D-hydroxybutyrate | Ketone bodies | −0.106 | 0.027 | 0.000126 |
| Creatine | Organic acid | −0.095 | 0.029 | 0.001101 |
| Lipid (mainly LDL, | Lipid | 0.087 | 0.028 | 0.002272 |
| fatty acid (CH₂)ₙ | | | | |
| moieties) | | | | |
| Lactate | Glycolysis | 0.067 | 0.025 | 0.007194 |
| | related | | | |
| Lysine | Amino acids | −0.060 | 0.027 | 0.025594 |
| Histidine | Amino acids | −0.057 | 0.027 | 0.036010 |
| Lipid (mainly VLDL, | Lipid | 0.043 | 0.023 | 0.061526 |
| fatty acid (CH₂) | | | | |
| moieties) | | | | |
| Dimethylglycine | Amino acids | −0.046 | 0.026 | 0.082072 |
| Glycine | Amino acids | 0.007 | 0.030 | 0.818118 |

TABLE 4
Estimates and p-values from mixed effects models
examining the association between metabolites and
insulin resistance in the main study (n = 150)

| | Metabolic | | | p-value |
|---|---|---|---|---|
| Metabolites | pathway | Coef | SE | (unadjusted) |
| Citrate | Glycolysis | −0.188 | 0.021 | <0.0001 |
| | related | | | |
| Phospholipids | Lipid | −0.066 | 0.024 | 0.0067 |
| 3-D-hydroxybutyrate | Ketone bodies | −0.092 | 0.018 | <0.0001 |
| Creatine | Organic acid | −0.142 | 0.023 | <0.0001 |
| Lipid (mainly LDL, | Lipid | 0.133 | 0.023 | <0.0001 |
| fatty acid (CH2)n | | | | |
| moieties) | | | | |
| Lactate | Glycolysis | 0.101 | 0.019 | <0.0001 |
| | related | | | |
| Lysine | Amino acids | −0.139 | 0.02 | <0.0001 |
| Histidine | Amino acids | −0.124 | 0.022 | <0.0001 |
| Lipid (mainly VLDL, | Lipid | −0.12 | 0.022 | <0.0001 |
| fatty acid (CH2) | | | | |
| moieties) | | | | |
| Dimethylglycine | Amino acids | −0.118 | 0.021 | <0.0001 |
| Glycine | Amino acids | −0.08 | 0.026 | 0.0023 |

The analysis has highlighted the importance of specific metabolites in amino acid, ketone body, glycolysis and fatty acid metabolism, in describing the variations of HOMA-IR throughout the childhood. This is believed to be the first report of a metabolic contribution of specific metabolic processes to overall insulin metabolism variations in a longitudinal and continuous manner.

### Central Energy-Related Metabolites

In the pilot and main study cohorts, mixed effects modelling described inverse associations of IR with citrate and 3-D-hydroxybutyrate overall (p<0.001), and positive associations of IR with lactate (p<0.01). The analysis of the main study describes statistically significant year-on-year correlations for citrate ranging from r=0.28 to r=0.66 p<0.05), while those for 3-D-hydroxybutyrate were not significant before 8 y and then ranged from r=0.21 to r=0.58 (p<0.05). In the main study, citrate was inversely correlated with IR at each cross-sectional time-point between 5 y and 16 y (correlations ranging from r=−0.21 to r=−0.52, p<0.05). 3-D-hydroxybutyrate showed inverse cross-sectional correlations (correlations ranging from r=−0.21 to r=−0.53, p<0.05) up to age. Lactate showed positive cross-sectional correlations with IR (correlations ranging from r=0.13 to r=0.45, p<0.05).

### Amino Add Metabolism

Mixed effects modelling identified statistically significant inverse associations between histidine, creatine and lysine with IR (p<0.05), which replicated in the main study (p<0.001). Each metabolite also showed inverse cross-sectional correlations with IR, particularly so between 9 y and 14 y (correlations ranging from r=−0.17 to r=−0.46, p<0.05).

Lipid Related Metabolites Associated with IR

¹H-NMR spectrum of human blood serum enables the monitoring of signals related to lipoprotein bound fatty acyl groups found in triglycerides, phospholipids and cholesteryl esters, together with peaks from the glyceryl moiety of triglycerides and the choline head group of phosphatidylcholine.

Here, signals derived from the methyl fatty acyl groups in phospholipids containing choline showed inverse associations with IR, whereas signals derived from the methyl fatty acyl groups in LDL particles showed positive associations with IR. The lipid signals were highly correlated with each other (r>0.8 between 5 y and 13 y, and r=0.6 at 14 y). These associations were also found in the main study both in the mixed effects model and at individual time-points. Cross-sectional associations between IR and phospholipids were inverse and statistically significant from age 7 y (correlations ranged from r=−0.19 to r=−0.54), whereas those between IR and fatty acyl groups in LDL particles were positive and statistically significant between 7 y and 14 y (correlations ranging from r=0.24 to r=0.41). Whilst not significant in the pilot study (p=0.06), fatty acyl groups in VLDL particles showed a positive association with IR in the mixed effects model, consistent with cross-sectional association which were positive and statistically significant at age 5 y and between 7 y and 14 y (correlations ranging from r=0.25 to r=0.46).

### Example 3

### Metabolites Indicative of Higher HOMA-IR at Adolescence

For each metabolite showing a significant association overtime with IR, the present inventors assessed further if their serum concentration was informative of low or high IR status over the 14-16 year age range. Arbitrarily the 91^{st} centile for the HOMA-IR distribution was employed as a threshold to define children with high IR status (Table 5). It was further explored—amongst the metabolites contributing the most to HOMA-IR variations in childhood—which ones may be an earlier and a more indicative indicator of higher HOMA-IR at adolescence.

TABLE 5
Estimates and p-values from mixed effects models examining
the association between metabolites and HOMA-IR groups

| | p-value | p-value |
|---|---|---|
| | Group | Age: group |
| Metabolites | difference | interaction |
| Citrate | <0.01 | NS |
| Phospholipids | <0.001 | NS |
| 3-D-hydroxybutyrate | <0.05 | NS |
| Lipid (mainly LDL, fatty acid | <0.01 | NS |
| (CH2)n moieties) | | |
| Lactate | <0.001 | NS |
| Histidine | <0.05 | NS |
| Lysine | <0.01 | NS |
| Lipid (mainly VLDL, fatty acid | <0.01 | NS |
| (CH2) moieties) | | |
| Creatine | 0.56 | <0.05 |
| Glycine | <0.01 | NS |
| Creatine:Glycine ratio | <0.05 | <0.001 |

Therefore, amongst the most influential biochemical species contributing to high HOMA-IR in childhood, the analysis indicates that:
Mixed effects modelling identified a significant positive association overtime between high IR status and lactate, fatty acyl groups in LDL and VLDL particles, and creatine:glycine ratio.
A significant negative association was found overtime between high IR status and citrate, histidine, 3-D-hydroxybutyrate, glycine, creatine, lysine and phospholipids.
Fat mass (waist circumference) was also a statistically significant variable increased in high IR group over time (p<0.001), with a significant interaction between age and group (p<0.001).

Significant increases in the annual incidence of both type 1 diabetes and type 2 diabetes among youths (aged 10 to 19 years old) in the United States have been recently reported by Mayer-Davis et al. (Incidence Trends of Type 1 and Type 2 Diabetes among Youths, 2002-2012, The New England Journal of Medicine, 376:1419-1429, 2017). It is well established that variations exist across racial and ethnic groups. As illustrated by Mayer-Davis et al, this includes high relative increases in the incidence of type 2 diabetes in racial and ethnic groups other than non-Hispanic whites in the USA as an example. Variation across demographic subgroups may reflect varying combinations of genetic, environmental, and behavioural factors that contribute to diabetes. Therefore, reference values should be generated accordingly for the proposed markers.

As an example in the present study cohort, fold of changes between groups are determined from the population, and provided at representative ages (Table 6).

TABLE 6
Fold of change (percentage) in high HOMA-IR
subjects compared to reference group

| Age | 8.00 | 9.00 | 10.00 | 11.00 | 12.00 | 13.00 | 14.00 | 15.00 | 16.00 |
|---|---|---|---|---|---|---|---|---|---|
| Lactate | 11.68 | 4.89 | 11.50 | 8.52 | 13.14 | 13.50 | 12.01 | 18.69 | 13.19 |
| Lysine | 1.32 | 0.52 | 6.13 | −2.77 | −4.62 | −0.02 | −6.82 | −3.61 | 2.47 |
| Citric acid | −4.66 | −7.23 | −5.50 | −7.28 | −6.13 | −6.66 | −16.67 | −2.42 | −1.07 |
| Creatine | −10.87 | 0.28 | −3.78 | −13.86 | −6.72 | −0.57 | −10.57 | −3.43 | 2.83 |
| Glycine | −3.83 | −5.92 | −7.94 | −4.48 | −10.02 | −11.62 | −15.75 | −10.49 | −6.74 |
| Histidine | 2.99 | −6.44 | −1.70 | −3.22 | −3.96 | −9.30 | −8.46 | −3.54 | −5.27 |

The population of children overweight or obese at age 5, remains and further developed excessive fat mass gain and body weight gain throughout puberty and adolescence, and have higher HOMA-IR than in other children. In particular, subjects in the 91^{st} centile of HOMA-IR at adolescence have a particularly marked lower histidine concentration in serum from the age of 9, which corresponded to the period where IR trajectories diverged between groups. They also show a higher body fat and central adiposity (waist circumference) throughout childhood. The status in histidine is negatively associated with C-reactive protein levels at each age for the Earlybird population.

Our results also describe a remodelling of circulating phospholipid species throughout childhood, growth and development. This is a phenomenon well documented in the field of IR and T2D (Szymanska, Bouwman et al. 2012), but not in childhood in relation growth, development and excess of fat gain. The remodelling of phospholipid species is often linked to decreased concentrations in ether-lipids (plasmalogens) in relation to oxidative stress, which have been reported in several diseases; e.g. diabetes mellitus, vascular diseases and obesity.

Histidine and lysine are two representative targets of oxidative modifications. Histidine is extremely sensitive to a metal-catalyzed oxidation, generating 2-oxo-histidine and its ring-ruptured products, whereas the oxidation of lysine generates carbonyl products, such as aminoadipic semialdehyde. On the other hand, both histidine and lysine are nucleophilic amino acids and therefore vulnerable to modification by lipid peroxidation derived electrophiles, such as 2-alkenals, 4-hydroxy-2-alkenals, and ketoaldehydes, derived from lipid peroxidation. Histidine shows specific reactivity toward 2-alkenals and 4-hydroxy-2-alkenals, whereas lysine is a ubiquitous target of aldehydes, generating various types of adducts. Covalent binding of reactive aldehydes to histidine and lysine is associated with the appearance of carbonyl reactivity and antigenicity of proteins. None of these amino acids are reported markers of IR in adult obese subjects. Histidine and arginine status were associated with inflammation and oxidative stress in obese adult women with metabolic syndrome (Niu, Feng et al. 2012). Furthermore, histidine supplementation is thought to improve IR by reducing inflammation in obese women with the metabolic syndrome (Feng, Niu et al. 2013).

Since inefficient lipolysis (high basal/low stimulated) was linked to future weight gain and impaired glucose metabolism and may constitute a treatment target (Arner, Andersson et al. 2018), our observation indicate distinctive nutritional requirements during growth and development to promote healthy fat metabolism. In the Earlybird cohort, overweight children at age 5 remain overweight throughout childhood, and will acquire a high IR status from age 10 during pubertal development and development of additional fat mass. Therefore, our observations of negative association with histidine, lysine, and arginine status may be indicative of potential deregulation of oxidative stress and adipocyte lipolysis during growth and development, which are concomitant or contributing to IR development.

### Example 4

### Amino Acid and HOMA-IR Status at Adolescence Links to HOMA-IR Status in Adulthood

For bath insulin and HOMA-IR, using spearman correlation analysis, the present inventors described how insulin and HOMA-II status in children and adolescents consistently associated statistically significantly with adult status from 11 years throughout childhood and adolescence (Table 7). Therefore, the metabolites contributing the most to HOMA-IR variations in childhood are more indicator of higher HOMA-IR in childhood are relevant markers for high HOMA-IR status in adulthood.

TABLE 7
Spearman Correlation coefficient between subject parameters in
childhood with parameters of the same subjects when aged 20

| Readout | | | | | | |
|---|---|---|---|---|---|---|
| (N = 141) | Age 16 | Age 15 | Age 14 | Age 13 | Age 12 | Age 11 |
| Insulin | 0.2208 | 0.1893 | 0.3109 | 0.2579 | 0.1894 | 0.2126 |
| | (p < 0.001) | (p < 0.05) | (p < 0.001) | (p < 0.001) | (p < 0.05) | (p < 0.01) |
| HOMA1 | 0.2366 | 0.1992 | 0.3142 | 0.2606 | 0.1904 | 0.2235 |
| IR | (p < 0.01) | (p < 0.05) | (p < 0.001) | (p < 0.001) | (p < 0.05) | (p < 0.01) |

Legend: Spearman correlation analysis, data as r coefficient, p value,
* 95% CI,
** 99% CI,
*** 99.9% CI

In addition, quantitative measures of amino acids were performed in serum samples from the same healthy subjects collected at year 15 and year 20, to provide guidance on healthy reference ranges.

TABLE 8
reference amino acid concentrations
in reference subject groups (N = 168)

Metabolite concentrations in micro molar
(Mean ± SD)

| Marker | Year 15 | Year 20 |
|---|---|---|
| Histidine | 92 ± 14 | 90 ± 15 |
| Lysine | 174 ± 39 | 177 ± 48 |
| Glycine | 304 ± 67 | 279 ± 64 |

Amed, S., N. Islam, J. Sutherland and K. Reimer (2018). “Incidence and prevalence trends of youth-onset type 2 diabetes in a cohort of Canadian youth: 2002-2013.” Pediatr Diabetes 19(4): 630-636.
Bates, D., M. Maechler, B. Bolker and S. Walker (2015). “Fitting Linear Mixed-Effects Models Using Ime4.” Journal of Statistical Software 67(1): 1-48.
Guasch-Ferre, M., A. Hruby, E. Toledo, C. B. Clish, M. A. Martinez-Gonzalez, J. Salas-Salvado and F. B. Hu (2016). “Metabolomics in Prediabetes and Diabetes: A Systematic Review and Meta-analysis.” Diabetes Care 39(5): 833-846.
Kuznetsova, A., P. B. Brockhoff and R. H. B. Christensen (2016). “ImerTest: Tests in Linear Mixed Effects Models. R package version 2.0-30. https://CRAN.R-project.org/package=ImerTest.”
Lauria, M., M. Persico, N. Dordevic, O. Cominetti, A. Matone, J. Hosking, A. Jeffery, J. Pinkney, L. Da Silva, C. Priami, I. Montoliu and F. P. Martin (2018). “Consensus Clustering of temporal profiles for the identification of metabolic markers of pre-diabetes in childhood (EarlyBird 73).” Sci Rep 8(1): 1393.
Martin, F. J., I. Montoliu and M. Kussmann (2017). “Metabonomics of ageing—Towards understanding metabolism of a long and healthy life.” Mech Ageing Dev 165(Pt B): 171-179.
Mayer-Davis, E. J., D. Dabelea and J. M. Lawrence (2017). “Incidence Trends of Type 1 and Type 2 Diabetes among Youths, 2002-2012.” N Engl J Med 377(3): 301.
Pinkney, J., A. Streeter, J. Hosking, M. Mohammod, A. Jeffery and T. Wilkin (2014). “Adiposity, chronic inflammation, and the prepubertal decline of sex hormone binding globulin in children: evidence for associations with the timing of puberty (Earlybird 58).” J Clin Endocrinol Metab 99(9): 3224-3232.
Riedl, A., C. Gieger, H. Hauner, H. Daniel and J. Linseisen (2017). “Metabotyping and its application in targeted nutrition: an overview.” Br J Nutr 117(12): 1631-1644.
Sperisen, P., O. Cominetti and F. P. Martin (2015). “Longitudinal omics modeling and integration in clinical metabonomics research: challenges in childhood metabolic health research.” Front Mol Biosci 2:44.
Zhao, X., X. Gang, Y. Liu, C. Sun, Q. Han and G. Wang (2016). “Using Metabolomic Profiles as Biomarkers for Insulin Resistance in Childhood Obesity: A Systematic Review.” J Diabetes Res 2016: 8160545.

1. A method for predicting insulin resistance in a subject, the method comprising:
a. (i) determining the levels of lactate and histidine, and one or more of citrate, 3-D-hydroxybutyrate, lysine, glycine, and creatine in a biofluid sample of the subject; or (ii) determining the levels of one or more of lactate, histidine, citrate, 3-D-hydroxybutyrate, lysine, glycine, and creatine in a biofluid sample of the subject;
b. comparing the levels of one or more of lactate, histidine, creatine:glycine ratio, citrate, 3-D-hydroxybutyrate, lysine with a reference value;
c. identifying the subject as being at high risk of insulin resistance if
(I) the levels of one or more of lactate, creatine:glycine ratio are higher than the reference value in b; or
(II) the levels of one or more of histidine, citrate, 3-D-hydroxybutyrate, lysine are lower than the reference value in b.

2. The method for predicting insulin resistance in a subject according to claim 1, the method comprising:
a. (i) determining the levels of lactate and histidine, and one or more of citrate, 3-D-hydroxybutyrate, lysine, glycine, and creatine in a biofluid sample collected from the subject being a child or an adolescent; or (ii) determining the levels of one or more of lactate, histidine, citrate, 3-D-hydroxybutyrate, lysine, glycine, and creatine in a biofluid sample collected from the subject being a child or an adolescent;
b. comparing the levels of one or more of lactate, histidine, creatine:glycine ratio, citrate, 3-D-hydroxybutyrate, lysine with a reference value;
c. identifying the subject as being at high risk of insulin resistance in adolescence and/or adulthood if
(I) the levels of one or more of lactate, creatine:glycine ratio are higher than the reference value in b; or
(II) the levels of one or more of histidine, citrate, 3-D-hydroxybutyrate, lysine are lower than the reference value in b.

3. The method for predicting insulin resistance in a subject according to claim 1, wherein the method comprises:
a. determining the level of lactate in the biofluid sample collected from the subject being a child or an adolescent;
b. comparing the level of lactate with a reference value;
c. identifying the subject as being at high risk of high insulin resistance in adolescence and/or adulthood if the level of lactate is higher than the reference value in b.

4. The method for predicting insulin resistance in a subject according to claim 1, wherein the method comprises:
a. determining the level of glycine and creatine in the biofluid sample collected from the subject being a child or an adolescent;
b. comparing the level of creatine:glycine ratio with a reference value;
c. identifying the subject as being at high risk of high insulin resistance in adolescence and/or adulthood if the level of creatine:glycine ratio is higher than the reference value in b.

5. The method for predicting insulin resistance in a subject according to claim 1, wherein the method comprises:
a. determining the level of histidine in the biofluid sample collected from the subject being a child or an adolescent;
b. comparing the level of lactate with a reference value;
c. identifying the subject as being at high risk of high insulin resistance in adolescence and/or adulthood if the level of histidine is higher than the reference value in b.

6. The method for predicting insulin resistance in a subject according to claim 1, wherein the levels of lactate, histidine, creatine, glycine and one or more of citrate, 3-D-hydroxybutyrate, lysine, in a biofluid sample collected from the subject in step a(i) are determined.

7. The method for predicting insulin resistance in a subject according to claim 1, wherein the levels of lactate, histidine, creatine, glycine and two or more of citrate, 3-D-hydroxybutyrate, lysine, in a biofluid sample collected from the subject in step a(i) are determined.

8. The method for predicting insulin resistance in a subject according to claim 1, wherein the levels of lactate, histidine, creatine, glycine, citrate, 3-D-hydroxybutyrate, lysine in a biofluid sample collected from the subject in step a(i) are determined.

9. The method for predicting insulin resistance in a subject according to claim 1, wherein the biofluid sample is collected from a child subject in step a (i) and the subject is identified as being at high risk of IR in adolescence in step c.

10. The method for predicting insulin resistance in a subject according to claim 1, wherein the biofluid sample is collected from a child subject in step a (i) and the subject is identified as being at high risk of insulin resistance in adulthood in step c.

11. The method for predicting insulin resistance in a subject according to claim 1, wherein the biofluid sample is collected from an adolescent subject in step a (i) and the subject is identified as being at high risk of insulin resistance in adolescence in step c.

12. The method for predicting insulin resistance in a subject according to claim 1, wherein the biofluid sample is collected from an adolescent subject in step a (i) and the subject is identified as being at high risk of IR in adulthood in step c.

13-16. (canceled)

17. The method for predicting insulin resistance in a subject according to claim 1, wherein the biofluid sample is human blood serum.

18. A method of improving glucose level management in a child or an adolescent subject comprising (i) predicting whether the subject has insulin resistance, the method comprising:
a. (i) determining the levels of lactate and histidine, and one or more of citrate, 3-D-hydroxybutyrate, lysine, glycine, and creatine in a biofluid sample of the subject; or (ii) determining the levels of one or more of lactate, histidine, citrate, 3-D-hydroxybutyrate, lysine, glycine, and creatine in a biofluid sample of the subject;
b. comparing the levels of one or more of lactate, histidine, creatine:glycine ratio, citrate, 3-D-hydroxybutyrate, lysine with a reference value;
c. identifying the subject as being at high risk of insulin resistance if
(I) the levels of one or more of lactate, creatine:glycine ratio are higher than the reference value in b; or
(II) the levels of one or more of histidine, citrate, 3-D-hydroxybutyrate, lysine are lower than the reference value in b; and (ii) providing a method of modifying the lifestyle of a subject identified as being at higher risk of having insulin resistance in adolescence and/or adulthood, wherein the dietary intervention enhances insulin sensitivity, lowers insulin resistance and/or reduces the glucose level.

19. A method of improving glucose level management in a child or an adolescent subject according to claim 18, wherein the modification of lifestyle lowers insulin resistance.

20. A method of improving glucose level management in a child or an adolescent subject according to claim 18, wherein said modification of lifestyle is provided through prepuberty and puberty.

21-22. (canceled)

23. A method of improving glucose level management in a child or an adolescent subject according to claim 18, wherein the modification in lifestyle in the subject comprises a change in diet.

24-25. (canceled)

26. A kit of parts comprising means to measure levels of one or more of lactate, histidine, citrate, 3-D-hydroxybutyrate, lysine, glycine, and creatine in a biofluid sample of a child or adolescent subject.

27. Use of a kit of parts according to claim 26, to predict a child or adolescent subject of having insulin resistance or developing pre-diabetes in adolescence and/or adulthood.
